(19) United States
(12) Patent Application Publication
(10) Pub. No.: US 2026/0256748 A1
(43) Pub. Date: Sep. 3, 2026

(54) LANIFIBRANOR FOR USE IN THE TREATMENT OF SPLANCHNIC VASODILATATION IN A PATIENT WITH A LIVER CONDITION

(71) Applicant: INVENTIVA, Daix (FR)

(72) Inventors: Guillaume WETTSTEIN, Daix (FR); Jean-Louis JUNIEN, Paris (FR); Sander LEFERE, Ghent (BE); Anneleen HELDENS, Ghent (BE); Anja GEERTS, Ghent (BE)

(73) Assignee: INVENTIVA, Daix (FR)

(21) Appl. No.: 19/489,944

(22) PCT Filed: Jun. 4, 2024
(86) PCT No.: PCT/EP2024/065324

§ 371 Date: Dec. 4, 2025

(30) Foreign Application Priority Data
Jun. 5, 2023 (EP) 23305892.4

(51) Int. Cl. A61K 31/428; A61P 1/00; A61P 1/16; A61P 9/00

(52) U.S. Cl. CPC A61K 31/428; A61P 1/00; A61P 1/16; A61P 9/00

(57) ABSTRACT

The present invention relates to the use of lanifibranor or a deuterated form thereof or a pharmaceutical acceptable salt thereof for the treatment of splanchnic vasodilatation in a patient with a liver condition.

### FIELD OF THE INVENTION

The present invention relates to a method of treating splanchnic vasodilatation in a patient with a liver condition, which method comprises administering lanifibranor or a deuterated form thereof or a pharmaceutical salt thereof to the patient.

### BACKGROUND OF THE INVENTION

Liver condition such as cirrhosis, Acute Liver Failure (ALF) or Acute on Chronic Liver Failure (ACLF) are end-stage diseases arising from multiple different hepatic and extra-hepatic acute and chronical injuries. These injuries primarily impair the liver functions but also have systemic complications leading to multiple organ failure. One common driver of these extra-hepatic complications and organ failure is the alteration of the systemic haemodynamics. This is characterized by a splanchnic vasodilation leading to the formation of portal-systemic collaterals and other systemic haemodynamic alterations leading to reduced total peripheral resistance and systemic hypotension. In addition, in case of hepatic disease leading to portal hypertension an increase in spleen size due to increase venous pressure causing congestive splenomegaly is observed. Finally, the systemic hypotension is responsible for renal dysfunction (one of the causes of ascites) as well as increase cardiac output and hepatic encephalopathy. In conclusion, splanchnic vasodilation leads to hyperdynamic circulation which will in turn contribute to the maintenance and aggravation of liver failure. It is consequently of importance when treating advanced liver disease to be able to target the splanchnic vasodilation on top of the intrahepatic resistance. From a clinical point of view, the hyperdynamic syndrome mainly caused by splanchnic vasodilation can be considered a prerequisite for the development of the multi-organ failure.

Treatments for splanchnic vasodilation are known in the art and include, but are not limited to liver transplantation, dialysis, insertion of a transjugular intrahepatic portosystemic shunt (TIPS) (to reduce blood pressure in the portal vein), hemodialysis, liver dialysis, intravenous albumin infusion, splanchnic vasoconstrictors (e.g. vasopressin, midodrine, somatostatin, ornipressin, terlipressin), albumin-bound membrane dialysis (e.g. molecular adsorbents recirculation system (MARS)), pentoxyfylline, acetylcysteine, and misoprostol. Treatment is currently limited to palliative care, which merely delays the eventual need for liver transplantation. The only effective treatment of liver condition is transplantation. The use of such transplants, however, is limited due to donor shortages, high cost, and the requirement for life-long immunosuppression. Thus, there is a clear need for alternative treatments for the treatment of splanchnic vasodilatation, in particular in patient with a liver condition. Lanifibranor {4-[1-(1,3-benzothiazol-6-ylsulfonyl)-5-chloroindol-2-yl]butanoic acid; CAS 927961-18-0} is a pan-PPAR agonist which is currently in clinical development for the treatment of patients with non-alcoholic steatohepatitis (NASH). U.S. Pat. No. 11,504,380 discloses a method for preventing a cirrhotic subject at risk of progressing from compensated stage to decompensated stage, the method comprising administering lanifibranor to the subject. It has now been found that lanifibranor can exert a vasoconstrictor effect in the splanchnic compartment in patients with a liver condition. In particular it has been found that administration of lanifibranor improves mesenteric blood flow, mesenteric wall thickness, and spleen weight in such patients as well as reducing the mesenteric vasculature expansion, leading to reduce splanchnic vasodilatation in patients with a liver condition.

### SUMMARY OF THE INVENTION

The present disclosure relates to lanifibranor or a deuterated form thereof or a pharmaceutical salt thereof, or a pharmaceutical composition comprising lanifibranor or a deuterated form thereof or a pharmaceutical salt thereof, for use in the treatment of splanchnic vasodilatation in a patient with a liver condition. In some embodiments, the liver condition is cirrhosis, acute liver failure, acute-on-chronic liver failure, hepatopulmonary syndrome, hepatorenal syndrome and hepatic encephalopathy. In some embodiments, the liver condition is cirrhosis, advantageously decompensated cirrhosis.

The present disclosure also relates to a method of treating splanchnic vasodilatation in a patient with a liver condition. In particular, the present disclosure relates to a method of treating splanchnic vasodilatation in a patient with a liver condition, the method comprising administering lanifibranor or a deuterated form thereof or a pharmaceutical salt thereof, or a pharmaceutical composition comprising lanifibranor or deuterated form thereof or a pharmaceutical salt thereof, to the patient.

### DESCRIPTION OF THE DRAWINGS

FIG. 1: Evaluation of the spleen weight in animals with Partial portal vein ligation (PPVL) treated or not with lanifibranor at 10 and 30 mg/kg.

FIG. 2: Evaluation of the mesenteric vascular wall thickness in animals with Partial portal vein ligation (PPVL) treated or not with lanifibranor at 10 and 30 mg/kg (quantification and pictures of the mesenteric vasculature by uCT).

FIG. 3: Evaluation of the flow superior mesenteric artery in animals with Partial portal vein ligation (PPVL) treated or not with lanifibranor at 10 and 30 mg/kg.

FIG. 4: Evaluation of the vasculature system using an immunohistochemistry staining for CD34 that is expressed on capillarized blood vessels in animals with Partial portal vein ligation (PPVL) treated or not with lanifibranor at 10 and 30 mg/kg.

### DETAILED DESCRIPTION OF THE INVENTION

As used herein, the articles including “a” and “an”, are understood to mean one or more of what is claimed or described.

As used herein the term “about xx” includes the value “xx”.

As used herein, the term “from xx to yy” includes the end points xx and yy.

As used herein, the term “prevent”, “prevention” or “prophylaxis” or “preventive treatment” or “prophylactic treatment” includes a treatment leading to the prevention of a disease as well as a treatment reducing and/or delaying the incidence of a disease or the risk of the disease occurring.

As used herein, the term “treatment” or “curative treatment” is defined as a treatment leading to a cure or a treatment which alleviates, improves and/or eliminates, reduces and/or stabilizes the symptoms of a disease or the suffering that it causes. According to the present invention, the term “for use in the treatment of splanchnic vasodilatation in a patient” or “for use in the curative treatment of splanchnic vasodilatation in a patient” means that the product used has a direct effect on splanchnic vasculature, and notably improves mesenteric blood flow, mesenteric wall thickness, spleen weight, and reduces mesenteric vasculature expansion.

As used herein, the term “splanchnic vasodilatation” refers to a widening of the vasculature supplying the intestines, e.g. by relaxation of the smooth muscle cells lining the vasculature. Splanchnic vasodilation includes venous splanchnic vasodilatation and/or arterial splanchnic vasodilatation.

The embodiments described herein can be combined.

The present disclosure relates to lanifibranor or a deuterated derivative thereof or a pharmaceutical salt thereof for use in the treatment of splanchnic vasodilatation in a patient with a liver condition.

Lanifibranor {4-[1-(1,3-benzothiazol-6-ylsulfonyl)-5-chloroindol-2-yl]butanoic acid; CAS 927961-18-0} is a pan-PPAR agonist. Lanifibranor is described in example 117 of WO 2007/026097, where it is obtained as a pale-yellow powder having a melting point of 74-80° C., and has the following structure:

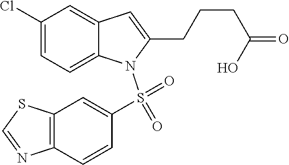 [SMILES: O=C(O)CCCc1cc2cc(Cl)ccc2n1S(=O)(=O)c1ccc2ncsc2c1]

In some embodiments, lanifibranor is in crystalline form. Examples of crystalline forms of lanifibranor are described in WO 2023/194339, WO 2023/016319, WO 2022/122014, WO 2022/261410 or WO 2022/258060, all incorporated herein by reference.

In some embodiments, lanifibranor can be a deuterated form of lanifibranor. In some embodiments, a deuterated form of lanifibranor is a compound of formula (I):

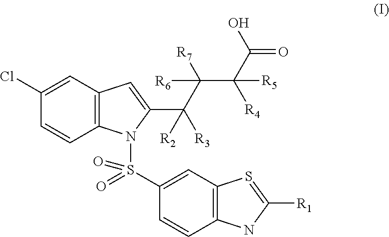

wherein at least one of the groups R₁ to R₇ is a deuterium (D) atom and the other groups R₁ to R₇ are hydrogen (H) atoms, as described in FR-A-3084254. In some aspects, at least group R₁ is D. In some aspects at least one of the groups R₂ to R₇ is D, notably at least one of the groups R₂ and R₃ and/or at least one of the groups R₄ and R₅ and/or at least one of the groups R₆ and R₇ is D. In a preferred aspect each of R₂, R₃, R₄, R₅, R₆ and R₇ is D.

In some embodiments, a deuterated form of lanifibranor is 4-(1-(2-deuterio-1,3-benzothiazol-6-yl)sulfonyl)-5-chloro-1H-indol-2-yl)butanoic acid. In other embodiments a deuterated form of lanifibranor is 4-[1-(1,3-benzothiazol-6-ylsulfonyl)-5-chloro-indol-2-yl]-2,2,3,3,4,4-hexadeuteriobutanoic acid.

In some embodiments, lanifibranor or a deuterated form thereof is in the form of one of its pharmaceutically acceptable salts or solvates. The term “solvate” is used herein to describe a molecular complex comprising lanifibranor or a deuterated form thereof and one or more pharmaceutically acceptable solvent molecules, for example, ethanol. The term “hydrate” is employed when said solvent is water. Pharmaceutically acceptable salts of lanifibranor or a deuterated form thereof include base addition salts thereof. Base addition salts may be prepared from inorganic and organic bases. Examples of inorganic bases include sodium hydroxide, potassium hydroxide, magnesium hydroxide and calcium hydroxide. Examples of organic bases include amines, amino alcohols, basic amino acids such as lysine or arginine, and quaternary ammonium compounds such as betaine or choline.

The inventors have shown that lanifibranor or a deuterated form thereof or a pharmaceutical acceptable salt thereof is particularly useful for treating splanchnic vasodilatation in patients with a liver condition. Indeed, lanifibranor has been tested in a mouse model in which the animals are free from a liver disorder or condition, in particular the animals are not cirrhotic. In this model, the portal vein of the animals has been partially ligated. Partial ligation of the portal vein leads to vasoconstriction (increase in portal pressure) without however inducing cirrhosis. In turn, vasoconstriction leads to a deterioration of splanchnic circulation. The model thus mimics conditions which can develop in patients with a liver condition, such as hepatic or extra-hepatic precipitating events. Administration of lanifibranor improved mesenteric blood flow, mesenteric wall thickness, spleen weight and reduced mesenteric vasculature expansion in mice. The used mice model is known to reflect prehepatic portal hypertension with splanchnic vasodilatation, hyperdynamics circulation and portal systemic shunts without hepatic impairment. The obtained data demonstrated a direct effect of lanifibranor on the splanchnic vasculature that is not consequent to a hepatic improvement. It therefore results that lanifibranor or a deuterated form thereof or a pharmaceutical salt thereof can exert curative effects, in particular a vasoconstrictor effect in the splanchnic vasculature, in patients with a liver condition.

In one embodiment the patient is a human. In another embodiment the patient is a non-human animal, e.g., a dog, cat, horse, cow, pig, sheep, goat or primate.

In some embodiments, the liver condition is cirrhosis, acute liver failure, acute-on-chronic liver failure, hepatopulmonary syndrome, hepatorenal syndrome and hepatic encephalopathy. In some embodiments, the liver condition is cirrhosis. In some embodiments, cirrhosis is decompensated cirrhosis.

As used herein, the term “Acute Liver Failure” (ALF) is defined as a syndrome of rapid decline in liver function characterized by jaundice, coagulopathy (INR>1.5) and hepatic encephalopathy in patients with no evidence of prior liver disease. Acute liver failure is a further condition where it is thought that non-apoptotic forms of cell death play an important role in disease progression and development, and thus present likely therapeutic targets. There are many causes of ALF including drug toxicity, drug overdose, paracetamol overdose, autoimmune hepatitis, viral hepatitis, Wilson's disease, etc.

As used herein, the term “acute-on-chronic liver failure” (ACLF) refers to a distinct clinical entity encompassing an acute deterioration of the liver function in patients with cirrhosis, often decompensated cirrhosis, which is usually associated with a precipitating event and results in the failure of one or more organs and high short-term mortality. Unregulated inflammation is thought to be a major contributing factor. A characteristic feature of ACLF is its rapid progression, the requirement for multiple organ supports and a high incidence of short- and medium-term mortality of 50-90%.

As used herein, the term “cirrhosis” includes cirrhosis caused by alcohol use disorder, such as early stage alcoholism, chronic alcoholism or end-stage alcoholism; cirrhosis caused by chronic viral hepatitis; cirrhosis caused by Non-Alcoholic Fatty Liver Disease (NAFLD), and/or Non-Alcoholic SteatoHepatitis (NASH), and/or metabolic-associated fatty liver disease (MAFLD), and/or metabolic-associated steatohepatitis (MASH); cirrhosis caused by primary biliary cirrhosis and/or primary sclerosing cholangitis or even cirrhosis caused by medication. In some embodiments, cirrhosis is compensated cirrhosis or decompensated cirrhosis. In some embodiments, decompensated cirrhosis includes decompensated cirrhosis caused by alcohol use disorder, such as early stage alcoholism, chronic alcoholism or end-stage alcoholism; decompensated cirrhosis caused by chronic viral hepatitis; decompensated cirrhosis caused by Non-Alcoholic Fatty Liver Disease (NAFLD), and/or Non-Alcoholic Steatohepatitis (NASH), and/or metabolic-associated fatty liver disease (MAFLD), and/or metabolic-associated steatohepatitis (MASH); decompensated cirrhosis caused by primary biliary cirrhosis and/or primary sclerosing cholangitis or even decompensated cirrhosis caused by medication. As used herein, “hepatorenal syndrome” (HRS) refers to a condition characterized by deterioration of the kidney function in patients with a liver disease (e.g. cirrhosis, alcoholic hepatitis or fulminant liver failure). HRS typically occurs when the liver suffers an acute injury, e.g. infection, bleeding in the gastrointestinal tract, and/or overuse of diuretic medications. Hepatorenal syndrome is characterized by congestion and blockage of intrahepatic microvasculature, causing portal hypertension.

As used herein, the term “hepatitis” refers to hepatitis A, B or C.

In some embodiments, the patient with a liver condition is already being treated for that condition. In some embodiments, the pre-existing treatment includes anti-diabetic agents, non-selective beta blockers, statins, anti-microbial agents, vasoconstrictor agents, lactulose therapy, or rifaximin.

Another aspect of the present disclosure relates to a pharmaceutical composition comprising a pharmaceutically effective amount of lanifibranor or a deuterated form thereof or a pharmaceutical salt thereof for use in the treatment of splanchnic vasodilatation in a patient with a liver condition. Advantageously, the pharmaceutical composition of the present disclosure comprises a pharmaceutically effective amount of lanifibranor or a deuterated form thereof or a pharmaceutical salt thereof as an active ingredient and one or more pharmaceutically acceptable excipients. In some embodiments, the present disclosure relates to a pharmaceutical composition consisting of a pharmaceutically effective amount of lanifibranor or a deuterated form thereof or a pharmaceutical salt thereof as an active ingredient and one or more pharmaceutically acceptable excipients. In some embodiments, lanifibranor (or a deuterated form thereof or a pharmaceutical salt thereof) is formulated into a pharmaceutical composition comprising one or more pharmaceutically acceptable excipients. Advantageously, the pharmaceutical composition comprising a therapeutically effective amount of lanifibranor or a deuterated form thereof or a pharmaceutical salt thereof as an active ingredient is administered to a patient suffering from or suspected of suffering from splanchnic vasodilatation.

According to embodiments that involve administering to a patient in need of treatment a therapeutically effective amount of lanifibranor or a deuterated form thereof or a pharmaceutical salt thereof, “therapeutically effective” or “an amount effective to treat” or “pharmaceutically effective” denotes the amount of lanifibranor, or a deuterated form thereof or a pharmaceutical salt thereof, or of a pharmaceutical composition needed to inhibit or reverse a disease condition (e.g., to treat vasodilatation splanchnic in a patient with a liver condition). Determining a therapeutically effective amount specifically depends on such factors as toxicity and efficacy of the medicament. These factors will differ depending on other factors such as potency, relative bioavailability, patient body weight, severity of adverse side-effects and preferred mode of administration. Toxicity may be determined using methods well known in the art. Efficacy may be determined utilizing the same guidance. A pharmaceutically effective amount, therefore, is an amount that is deemed by the clinician to be toxicologically tolerable, yet efficacious.

Dosage may be adjusted appropriately to achieve desired lanifibranor (or a deuterated form thereof) level, local or systemic, depending upon the mode of administration. In the event that the response in a patient is insufficient at such doses, even higher doses (or effective higher doses by a different, more localized delivery route) may be employed to the extent that patient tolerance permits. Multiple doses per day may also be employed to achieve appropriate systemic levels of lanifibranor or a pharmaceutically salt thereof. Appropriate systemic levels can be determined by, for example, measurement of the patient's peak or sustained plasma level of the drug. “Dose” and “dosage” are used interchangeably herein.

In some embodiments, lanifibranor or a deuterated form thereof or a pharmaceutical salt thereof is administered at a dose of from about 5 mg to about 1,200 mg. Advantageously, lanifibranor or a deuterated form thereof or a pharmaceutical salt thereof is administered at least at a dose of about 5 mg, at least at a dose of about 10 mg, at least at a dose of about 15 mg, at least at a dose of about 20 mg, at least at a dose of about 25 mg, at least at a dose of about 30 mg, at least at a dose of about 35 mg, at least at a dose of about 40 mg, at least at a dose of about 45 mg, at least at a dose of about 50 mg, at least at a dose of about 55 mg, at least at a dose of about 60 mg, at least at a dose of about 65 mg, at least at a dose of about 70 mg, at least at a dose of about 75 mg, at least at a dose of about 80 mg, at least at a dose of about 85 mg, at least at a dose of about 90 mg, at least at a dose of about 95 mg, at least at a dose of about 100 mg, at least at a dose of about 150 mg, at least at a dose of about 200 mg, at least at a dose of about 250 mg, at least at a dose of about 300 mg, at least at a dose of about 350 mg, at least at a dose of about 400 mg, at least at a dose of about 450 mg, at least at a dose of about 500 mg, at least at a dose of about 550 mg, at least at a dose of about 600 mg, at least at a dose of about 650 mg, at least at a dose of about 700 mg, at least at a dose of about 750 mg, at least at a dose of about at a dose of about 800 mg, at least at a dose of about 850 mg, at least at a dose of about 900 mg, at least at a dose of about 950 mg, at least at a dose of about 1,000 mg, at least at a dose of about 1,050 mg, at least at a dose of about 1,100 mg, at least at a dose of about 1,150 mg, or at a dose of about 1,200 mg. Lanifibranor (or a deuterated derivative thereof) can be administered once daily (“QD”), twice daily (“BID”), three time daily (“TID”) or four times daily (“QID”) provided the daily dose does not exceed the maximum amount indicated herein, i.e. about 1,200 mg. In some embodiments, lanifibranor (or a deuterated form thereof or a pharmaceutical salt thereof) is administered to a subject with a meal. In some embodiments, lanifibranor (or a deuterated form thereof or a pharmaceutical salt thereof) is administered to a subject under fasted conditions. In some embodiments, lanifibranor or a deuterated form thereof or a pharmaceutical salt thereof is administered at a daily dosage of from about 5 mg to about 1,200 mg. Advantageously, lanifibranor or a deuterated form thereof or a pharmaceutical salt thereof is administered at least at a daily dose of about 5 mg, at least at a daily dose of about 10 mg, at least at a daily dose of about 15 mg, at least at a daily dose of about 20 mg, at least at a daily dose of about 25 mg, at least at a daily dose of about 30 mg, at least at a daily dose of about 35 mg, at least at a daily dose of about 40 mg, at least at a daily dose of about 45 mg, at least at a daily dose of about 50 mg, at least at a daily dose of about 55 mg, at least at a daily dose of about 60 mg, at least at a daily dose of about 65 mg, at least at a daily dose of about 70 mg, at least at a daily dose of about 75 mg, at least at a daily dose of about 80 mg, at least at a daily dose of about 85 mg, at least at a daily dose of about 90 mg, at least at a daily dose of about 95 mg, at least at a daily dose of about 100 mg, at least at a daily dose of about 150 mg, at least at a daily dose of about 200 mg, at least at a daily dose of about 250 mg, at least at a daily dose of about 300 mg, at least at a daily dose of about 350 mg, at least at a daily dose of about 400 mg, at least at a daily dose of about 450 mg, at least at a daily dose of about 500 mg, at least at a daily dose of about 550 mg, at least at a daily dose of about 600 mg, at least at a daily dose of about 650 mg, at least at a daily dose of about 700 mg, at least at a daily dose of about 750 mg, at least at a daily dose of about at a daily dose of about 800 mg, at least at a daily dose of about 850 mg, at least at a daily dose of about 900 mg, at least at a daily dose of about 950 mg, at least at a daily dose of about 1,000 mg, at least at a daily dose of about 1,050 mg, at least at a daily dose of about 1,100 mg, at least at a daily dose of about 1,150 mg, or at a daily dose of about 1,200 mg.

In some embodiments, the compositions provided are employed for in vivo applications. Depending on the intended mode of administration in vivo the compositions used may be in a solid dosage form, a semi-solid dosage form or a liquid dosage form, the list being not limitative. In some embodiments, the pharmaceutical composition is a solid dosage form.

Exemplary solid dosage forms include tablets, capsules, stick-packs, sachets, lozenges, powders, pills, or granules. Preferred solid dosage forms include tablets, capsules and stick-packs, tablets being especially preferred. Advantageously, the compositions are administered in unit dosage forms suitable for single administration of precise dosage amounts. The compositions may also include, depending on the formulation desired, one or more pharmaceutically acceptable excipient(s). The choice of excipient(s) will to a large extent depend on factors such as the particular mode of administration, the effect of the excipient on solubility and stability, and the nature of the dosage form. Pharmaceutical compositions of the invention can be prepared by conventional methods, as described e.g. in Remington's Pharmaceutical Sciences, 19th Edition (Mack Publishing Company, 1995), incorporated herein by reference.

In some embodiments, the pharmaceutical composition comprises from 5 mg to 1,200 mg of lanifibranor or a deuterated form thereof or a pharmaceutical acceptable salt thereof. Exemplary pharmaceutical compositions comprise at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 150 mg, at least 200 mg, at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, at least 950 mg, at least 1,000 mg, at least 1,050 mg, at least 1,100 mg, at least 1,150 mg, or 1,200 mg of lanifibranor or a deuterated form thereof or a pharmaceutical acceptable salt thereof. Exemplary pharmaceutical compositions comprise 5 mg, 10 mg, 15 mg, 20 mg, 25 mg, 30 mg, 35 mg, 40 mg, 45 mg, 50 mg, 55 mg, 60 mg, at 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,050 mg, 1,100 mg, 1,150 mg, or 1,200 mg of lanifibranor or a deuterated form thereof or a pharmaceutical acceptable salt thereof.

In any of the embodiments described above, lanifibranor or a deuterated form thereof or a pharmaceutical acceptable salt thereof either per se or when present in a pharmaceutical composition, can be in crystalline form.

Administration during in vivo treatment may be by any routes, including oral, parenteral, intramuscular, intranasal, sublingual, intratracheal, inhalation, ocular, vaginal, and rectal. The skilled artisan will recognize that the route of administration may vary depending on the disorder to be treated. Advantageously, lanifibranor or a deuterated form thereof or a pharmaceutical acceptable salt thereof, or the pharmaceutical composition comprising lanifibranor or a deuterated form thereof or a pharmaceutical acceptable salt thereof may be administered to a patient via oral, parenteral or topical route. In some embodiments, the pharmaceutical compositions comprising lanifibranor or a deuterated form thereof or a pharmaceutical acceptable salt thereof are administered by oral route.

In some embodiments, the pharmaceutical composition comprising lanifibranor or a deuterated form thereof or a pharmaceutical acceptable salt thereof can further comprise a second active ingredient, such as empagliflozin, firsocostat, resmetirom, efruxifermin, belapectin, ocaliva, vasopressin, midodrine, somatostatin, ornipressin, terlipressin, pentoxyfylline, acetylcysteine, misoprostol, an anti-diabetic agent, a non-selective beta blocker, a statin, an anti-microbial agent, a vasoconstrictor agent, lactulose therapy, rifaximin or any suitable ingredient being effective in the treatment of such diseases.

Another aspect of the present disclosure relates to a method for the curative treatment of splanchnic vasodilatation in a patient with a liver condition, the method comprising administering an effective amount of lanifibranor or a deuterated form thereof or a pharmaceutical salt thereof, or of a pharmaceutical composition comprising an effective amount of lanifibranor or a deuterated form thereof or a pharmaceutical salt thereof, to the patient.

According to the present disclosure, lanifibranor or a deuterated form thereof or a pharmaceutical acceptable salt thereof, or a pharmaceutical composition comprising lanifibranor or a deuterated form thereof or a pharmaceutical acceptable salt thereof is particularly effective in treating a liver condition such as cirrhosis, acute liver failure, acute-on-chronic liver failure, hepatopulmonary syndrome, hepatorenal syndrome, and hepatic encephalopathy. Advantageously, lanifibranor or a deuterated form thereof or a pharmaceutical acceptable salt thereof, or a pharmaceutical composition comprising lanifibranor or a deuterated form thereof or a pharmaceutical acceptable salt thereof is particularly useful to treat splanchnic vasodilatation.

In some embodiments, the present disclosure relates to a method for the curative treatment of splanchnic vasodilatation in a patient with a liver condition, wherein the liver condition is chosen among cirrhosis, acute liver failure, acute-on-chronic liver failure, hepatopulmonary syndrome, hepatorenal syndrome and hepatic encephalopathy, the method comprising administering to the patient an effective amount of lanifibranor or a deuterated form thereof or a pharmaceutical acceptable salt thereof, or a pharmaceutical composition comprising an effective amount of lanifibranor or a deuterated form thereof or a pharmaceutical acceptable salt thereof as active ingredient and one or more pharmaceutically acceptable excipient(s) as defined above.

In some embodiments, the present disclosure relates to a method for the curative treatment of splanchnic vasodilatation in a patient with a liver condition, wherein the liver condition is cirrhosis, in particular decompensated cirrhosis, comprising administering to the patient an effective amount of lanifibranor or a deuterated form thereof or a pharmaceutical acceptable salt thereof, or a pharmaceutical composition comprising an effective amount of lanifibranor or a deuterated form thereof or a pharmaceutical acceptable salt thereof as active ingredient and one or more pharmaceutically acceptable excipient(s) as defined above.

In case of curative treatment, dosage, the administration mode and formulation as defined above are applicable.

Another aspect of the present disclosure relates to a method of treating splanchnic vasodilatation in a patient with a liver condition, the method comprising administering lanifibranor or a deuterated form thereof or a pharmaceutical salt thereof, or a pharmaceutical composition comprising lanifibranor or a deuterated form thereof or a pharmaceutical salt thereof, to the patient. Embodiments disclosed in relation to the methods described above also apply to this aspect of the disclosure.

In some embodiments, the present disclosure relates to a method of or treating splanchnic vasodilatation in a patient with a liver condition, comprising administering to the patient an effective amount of lanifibranor or a deuterated form thereof or a pharmaceutical acceptable salt thereof, or a pharmaceutical composition comprising an effective amount of lanifibranor or a deuterated form thereof or a pharmaceutical acceptable salt thereof as active ingredient and one or more pharmaceutically acceptable excipient(s) as defined above.

In some embodiments, the present disclosure relates to a method of treating splanchnic vasodilatation in a patient with acute liver failure, comprising administering to the patient an effective amount of lanifibranor or a deuterated form thereof or a pharmaceutical acceptable salt thereof, or a pharmaceutical composition comprising an effective amount of lanifibranor or a deuterated form thereof or a pharmaceutical acceptable salt thereof as active ingredient and one or more pharmaceutically acceptable excipient(s) as defined above.

In some embodiments, the present disclosure relates to a method of treating splanchnic vasodilatation in a patient with acute-on-chronic liver failure, comprising administering to the patient an effective amount of lanifibranor or a deuterated form thereof or a pharmaceutical acceptable salt thereof, or a pharmaceutical composition comprising an effective amount of lanifibranor or a deuterated form thereof or a pharmaceutical acceptable salt thereof as active ingredient and one or more pharmaceutically acceptable excipient(s) as defined above. Embodiments disclosed in relation to the methods described above also apply to this aspect of the disclosure.

In some embodiments, the present disclosure relates to a method of treating splanchnic vasodilatation in a patient with an hepatopulmonary syndrome, comprising administering to the patient an effective amount of lanifibranor or a deuterated form thereof or a pharmaceutical acceptable salt thereof, or a pharmaceutical composition comprising an effective amount of lanifibranor or a deuterated form thereof or a pharmaceutical acceptable salt thereof as active ingredient and one or more pharmaceutically acceptable excipient(s) as defined above.

Embodiments disclosed in relation to the methods described above also apply to this aspect of the disclosure.

In some embodiments, the present disclosure relates to a method of treating splanchnic vasodilatation in a patient with an hepatorenal syndrome, comprising administering to the patient an effective amount of lanifibranor or a deuterated form thereof or a pharmaceutical acceptable salt thereof, or a pharmaceutical composition comprising an effective amount of lanifibranor or a deuterated form thereof or a pharmaceutical acceptable salt thereof as active ingredient and one or more pharmaceutically acceptable excipient(s) as defined above. Embodiments disclosed in relation to the methods described above also apply to this aspect of the disclosure.

In some embodiments, the present disclosure relates to a method of treating splanchnic vasodilatation in a patient with an hepatic encephalopathy, comprising administering to the patient an effective amount of lanifibranor or a deuterated form thereof or a pharmaceutical acceptable salt thereof, or a pharmaceutical composition comprising an effective amount of lanifibranor or a deuterated form thereof or a pharmaceutical acceptable salt thereof as active ingredient and one or more pharmaceutically acceptable excipient(s) as defined above. Embodiments disclosed in relation to the methods described above also apply to this aspect of the disclosure.

In some embodiments, the present disclosure relates to a method of treating splanchnic vasodilatation in a patient with cirrhosis, comprising administering to the patient an effective amount of lanifibranor or a deuterated form thereof or a pharmaceutical acceptable salt thereof, or a pharmaceutical composition comprising an effective amount of lanifibranor or a deuterated form thereof or a pharmaceutical acceptable salt thereof as active ingredient and one or more pharmaceutically acceptable excipient(s) as defined above. In some embodiments, cirrhosis is caused by alcohol use disorder, such as early stage alcoholism, chronic alcoholism or end-stage alcoholism; by chronic viral hepatitis; by Non Alcoholic Fatty Liver Disease (NAFLD) and/or Non Alcoholic SteatoHepatitis (NASH); by primary biliary cirrhosis and/or primary sclerosing cholangitis or even by medication. In some embodiments, cirrhosis is compensated cirrhosis or decompensated cirrhosis. Embodiments disclosed in relation to the methods described above also apply to this aspect of the disclosure.

Another aspect of the present disclosure relates to the use of lanifibranor or a deuterated form thereof or a pharmaceutical acceptable salt thereof as defined above in the manufacture of a medicament for treating a liver condition, in particular for treating cirrhosis, acute liver failure, acute-on-chronic liver failure, hepatopulmonary syndrome, hepatorenal syndrome or hepatic encephalopathy.

Another aspect of the present disclosure relates to the use of lanifibranor or a deuterated form thereof or a pharmaceutical acceptable salt thereof as defined above in the manufacture of a medicament for treating cirrhosis, in particular for treating decompensated cirrhosis.

Embodiments disclosed in relation to the methods described above also apply to this aspect of the disclosure.

Another aspect of the present disclosure relates to the use of an effective amount of lanifibranor or a deuterated form thereof or a pharmaceutical acceptable salt thereof, as defined above, in the manufacture of a medicament for treating a liver condition, in particular for treating cirrhosis, acute liver failure, acute-on-chronic liver failure, hepatopulmonary syndrome, hepatorenal syndrome, or hepatic encephalopathy. Another aspect of the present disclosure relates to the use of an effective amount of lanifibranor or a deuterated form thereof or a pharmaceutical acceptable salt thereof, as defined above, in the manufacture of a medicament for treating cirrhosis, in particular for treating decompensated cirrhosis. Embodiments disclosed in relation to the methods described above also apply to this aspect of the disclosure. Another aspect of the present disclosure relates to the use of a pharmaceutical composition comprising an effective amount of lanifibranor or a deuterated form thereof or a pharmaceutical acceptable salt thereof as active ingredient and one or more pharmaceutically acceptable excipient(s) as defined above in the manufacture of a medicament for treating a liver condition, in particular for treating cirrhosis, acute liver failure, acute-on-chronic liver failure, hepatopulmonary syndrome, hepatorenal syndrome or hepatic encephalopathy. Another aspect of the present disclosure relates to the use of a pharmaceutical composition comprising an effective amount of lanifibranor or a deuterated form thereof or a pharmaceutical acceptable salt thereof as active ingredient and one or more pharmaceutically acceptable excipient(s) as defined above in the manufacture of a medicament for treating cirrhosis, in particular for treating decompensated cirrhosis. Embodiments disclosed in relation to the methods described above also apply to this aspect of the disclosure.

Aspects of the present disclosure are further illustrated by reference to the following, non-limiting embodiments.
1. A method of treating splanchnic vasodilatation in a patient with a liver condition, the method comprising administering lanifibranor or a deuterated form thereof or a pharmaceutical acceptable salt thereof, or a pharmaceutical composition comprising lanifibranor or a deuterated form thereof or a pharmaceutical acceptable salt thereof, to the patient.
2. A method of treating splanchnic vasodilatation in a patient with a liver condition according to item 1, wherein the liver condition is cirrhosis, acute liver failure, acute-on-chronic liver failure, hepatopulmonary syndrome, hepatorenal syndrome or hepatic encephalopathy.
3. A method of treating splanchnic vasodilatation in a patient with a liver condition according to item 2, wherein the liver condition is cirrhosis, in particular decompensated cirrhosis.
4. A method of treating splanchnic vasodilatation in a patient with a liver condition according to any of items 1 to 3, wherein the administration of lanifibranor, a deuterated form thereof or a pharmaceutical acceptable salt thereof, or of a pharmaceutical composition comprising lanifibranor or a deuterated form thereof or a pharmaceutical acceptable salt thereof, improves mesenteric blood flow, mesenteric wall thickness and/or spleen weight in the patient.
5. A method of treating splanchnic vasodilatation in a patient with a liver condition according to any of items 1 to 4, wherein lanifibranor or a deuterated form thereof or a pharmaceutical acceptable salt thereof is administered at a daily dose of from about 5 mg to about 1,200 mg.
6. A method of treating splanchnic vasodilatation in a patient with a liver condition according to any of items 1 to 5, wherein the pharmaceutical composition is a solid dosage form.
7. A method of treating splanchnic vasodilatation in a patient with a liver condition according to any of items 1 to 6, wherein the solid dosage form is a tablet, a capsule or a stick-pack.
8. A method of treating splanchnic vasodilatation in a patient with a liver condition according to any of items 1 to 7, wherein the pharmaceutical composition comprises from 5 mg to 1,200 mg of lanifibranor or a deuterated form thereof or a pharmaceutical acceptable salt thereof.
9. A method of treating splanchnic vasodilatation in a patient with a liver condition according to any of items 1 to 8, wherein the pharmaceutical composition comprises at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 150 mg, at least 200 mg, at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, at least 950 mg, at least 1,000 mg, at least 1,050 mg, at least 1,100 mg, at least 1,150 mg, or 1,200 mg of lanifibranor or a deuterated form thereof or a pharmaceutical acceptable salt thereof.
10. A method of treating splanchnic vasodilatation in a patient with decompensated cirrhosis, the method comprising administering lanifibranor or a deuterated form thereof or a pharmaceutical acceptable salt thereof, or a pharmaceutical composition comprising lanifibranor or a deuterated form thereof or a pharmaceutical acceptable salt thereof, to the patient.
11. A method of treating splanchnic vasodilatation in a patient with decompensated cirrhosis according to item 10, wherein the administration of lanifibranor, a deuterated form thereof or a pharmaceutical acceptable salt thereof, or of a pharmaceutical composition comprising lanifibranor or a deuterated form thereof or a pharmaceutical acceptable salt thereof, improves mesenteric blood flow, mesenteric wall thickness and/or spleen weight in the patient.
12. A method of treating splanchnic vasodilatation in a patient with decompensated cirrhosis according to any of items 10 to 11, wherein lanifibranor or a deuterated form thereof or a pharmaceutical acceptable salt thereof is administered at a daily dose of from about 5 mg to about 1,200 mg.
13. A method of treating splanchnic vasodilatation in a patient with decompensated cirrhosis according to any of items 10 to 12, wherein the pharmaceutical composition is a solid dosage form.
14. A method of treating splanchnic vasodilatation in a patient with decompensated cirrhosis according to any of items 10 to 13, wherein the solid dosage form is a tablet, a capsule or a stick-pack.
15. A method of treating splanchnic vasodilatation in a patient with decompensated cirrhosis according to any of items 10 to 14, wherein the pharmaceutical composition comprises from 5 mg to 1,200 mg of lanifibranor or a deuterated form thereof or a pharmaceutical acceptable salt thereof.
16. A method of treating splanchnic vasodilatation in a patient with decompensated cirrhosis according to any of items 10 to 15, wherein the pharmaceutical composition comprises at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 150 mg, at least 200 mg, at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, at least 950 mg, at least 1,000 mg, at least 1,050 mg, at least 1,100 mg, at least 1,150 mg, or 1,200 mg of lanifibranor or a deuterated form thereof or a pharmaceutical acceptable salt thereof.

The invention is illustrated by the following example.

### EXAMPLE

### Materials and Methods

Experiments were carried out using Swiss mice (7 to 10 animals per groups). Portal hypertension was induced using the partial portal vein ligation (PPVL) model. A midline abdominal incision was performed and the portal vein was separated from the surrounding tissue. A ligature (silk cut 5-0) was tied around both portal vein and adjacent 27-gauge blunt-tipped needle. Subsequent removal of the needle yielded a calibrated stenosis of the portal vein. One week after the ligation mice received daily lanifibranor (10 mg/kg or 30 mg/kg) or vehicle in a therapeutic setting for 7 days. The effect of lanifibranor on angiogenesis was evaluated by analyzing hepatic and systemic hemodynamics, serum, hepatic and mesenteric histology. The vascular corrosion casting is a technic allowing the capture of the 3D structure of the vascular bed. Briefly the vascular corrosion casts were obtained by inserting, after euthanasia, a 26-gauge catheter into the ileocolic vein, which was then perfused with Batson n°17 solution. The soft tissue was dissolved overnight in a 25% potassium hydroxide solution. The vascular corrosion casts of the venous mesenteric vasculature were analyzed using μCT.

### Results

### Hemodynamic Parameters

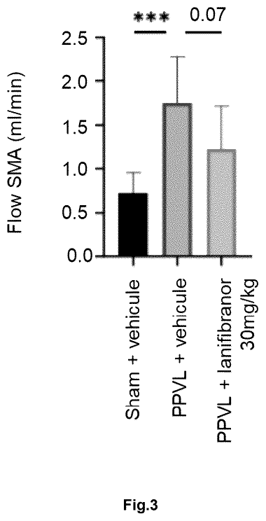

Mice with PPVL demonstrated a significant and high increase in portal pressure in comparison to control animals (from 4.34 for control to 10.85 mmHg for PPVL mice). Mice with PPVL also demonstrated a significant increase in flow superior mesenteric artery that was reduced by lanifibranor treatment at 30 mg/kg (reduction of 51.7%), as can be seen from FIG. 3.

### Spleen Weight

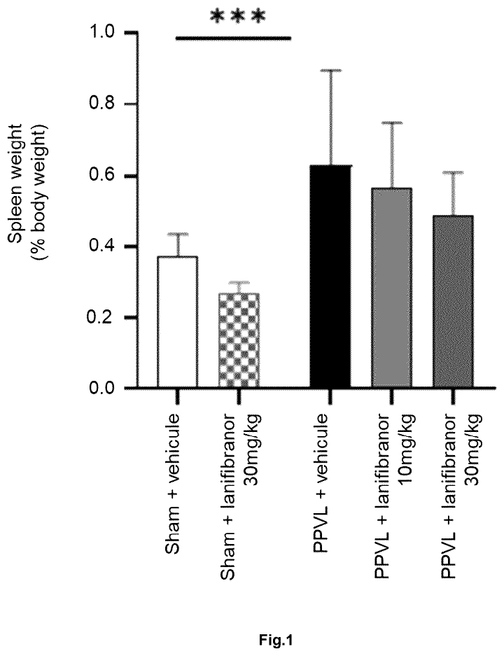

The spleen weight was significantly increased in mice with PPVL in comparison to control animals (from 0.38% body weight for control to 0.63% body weight for PPVL mice) due to the presence of portal hypertension. As can be seen from FIG. 1, lanifibranor dose-dependently reduced spleen weight by 23.7% (0.57% body weight at 10 mg/kg) and 56.1% (0.49% body weight at 30 mg/kg), demonstrating the improvement of mesenteric vasculature.

### Corrosion Vascular Casting

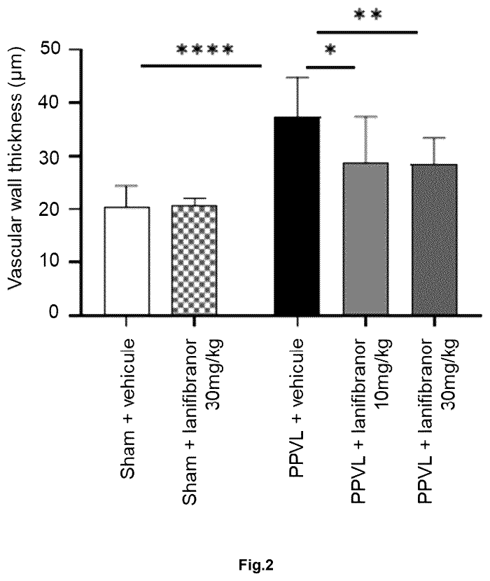

Evaluation by μCT of the vasculature showed an expansion of the venous mesenteric vasculature and increased mesenteric vascular wall thickness in mice with PPVL in comparison to control mice (from 20.31 μM for control to 37.53 μM for PPVL mice). As can be seen from FIG. 2, mesenteric vascular wall thickness was reduced by lanifibranor either at 10 mg/kg (28.95 μM, 49.8% reduction) or at 30 mg/kg (28.53 μM, 52.3% reduction). Expansion of the venous mesenteric vasculature was also reduced (data not shown).

### Angiogenesis and Vascular Expansion

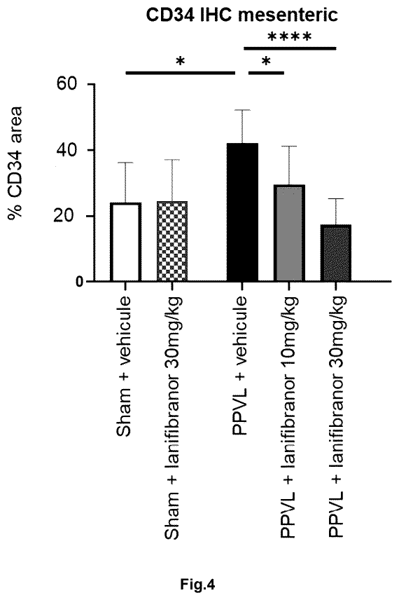

Evaluation of the vasculature system with an immunohistochemistry staining for CD34 allow to highlight the increase of blood vessels in the mesenteric area in PPVL animals. This reflect angiogenesis in this area due to increase mesenteric blood flow and portal pressure. This strength the data observed with the corrosion vascular casting showing an increase in the venous mesenteric vasculature. As observed with the corrosion casting technic the staining for CD34 significantly decreased in a dose dependent manner upon lanifibranor treatment (69.5% and 135.8% of reduction at 10 and 30 mg/kg of lanifibranor respectively) demonstrating the direct inhibition of angiogenesis and vascular expansion (FIG. 4).

1-11. (canceled)

12. A method of treating splanchnic vasodilatation in a patient with a liver condition, the method comprising administering lanifibranor or a deuterated form thereof or a pharmaceutical acceptable salt thereof, or a pharmaceutical composition comprising lanifibranor or a deuterated form thereof or a pharmaceutical acceptable salt thereof, to the patient.

13. The method of claim 12, wherein the liver condition is cirrhosis, acute liver failure, acute-on-chronic liver failure, hepatopulmonary syndrome, hepatorenal syndrome or hepatic encephalopathy.

14. The method of claim 13, wherein the liver condition is cirrhosis.

15. The method of claim 12, wherein lanifibranor or a deuterated form thereof or a pharmaceutical acceptable salt thereof is administered at a daily dose of from about 5 mg to about 1,200 mg.

16. The method of any of claims 12 to 15 claim 12, wherein the pharmaceutical composition is a solid dosage form.

17. The method of claim 16, wherein the solid dosage form is a tablet, a capsule or a stick-pack.

18. The method of claim 12, wherein the deuterated form of lanifibranor is a compound of formula (I): 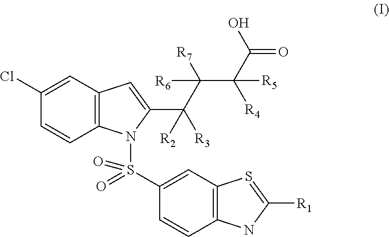
wherein at least one of the groups R₁ to R₇ is a deuterium (D) atom and the other groups R₁ to R₇ are hydrogen (H) atoms.

19. The method of claim 12, wherein the deuterated form of lanifibranor is 4-(1-(2-deuterio-1,3-benzothiazol-6-yl)sulfonyl-5-chloro-1H-indol-2-yl)butanoic acid or 4-[1-(1,3-benzothiazol-6-ylsulfonyl)-5-chloro-indol-2-yl]-2,2,3,3,4,4-hexadeuteriobutanoic acid.

20. The method of claim 12, wherein the liver condition is decompensated cirrhosis.

21. The method of claim 12, wherein the pharmaceutical composition comprises at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 150 mg, at least 200 mg, at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, at least 950 mg, at least 1,000 mg, at least 1,050 mg, at least 1,100 mg, at least 1,150 mg, or 1,200 mg of lanifibranor or a deuterated form thereof or a pharmaceutical acceptable salt thereof.

22. The method of claim 12, wherein the pharmaceutical composition comprises 400 mg, 600 mg or 800 mg of lanifibranor or a deuterated form thereof or a pharmaceutical acceptable salt thereof.

23. The method of claim 12, wherein the pharmaceutical is administered via oral, parenteral or topical route.

24. The method of claim 12, wherein the solid dosage form is a sachet, a lozenge, a powder, a pill or a granule.
